(19) United States
(12) Patent Application Publication
(10) Pub. No.: US 2025/0213833 A1
(43) Pub. Date: Jul. 3, 2025

(54) HUB ADAPTER FOR COUPLING MEDICAL DEVICES

(71) Applicant: Boston Scientific Medical Device Limited, Galway (IE)

(72) Inventors: Charlene Leung, Scarborough (CA); Gareth Davies, Toronto (CA)

(21) Appl. No.: 18/990,992

(22) Filed: Dec. 20, 2024

Related U.S. Application Data
(60) Provisional application No. 63/616,201, filed on Dec. 29, 2023.

(51) Int. Cl. A61M 29/00; A61M 39/10

(52) U.S. Cl. CPC A61M 29/00; A61M 39/1011

(57) ABSTRACT

An adaptable dilator for use with a crossing device is provided herein. The dilator comprises a dilator hub housing defining a distal end and a proximal end. the dilator further comprises a dilator body extending distally from the dilator hub housing. The dilator further comprises a locking feature disposed adjacent the proximal side of the dilator hub housing. The locking feature being movable between a neutral position defining a first diameter and a second position defining a second diameter. The locking feature also defines an engaged position defining an engaged diameter, the engaged position between the neutral position and the second position and the engaged diameter between the first diameter and the second diameter. The engaged diameter is variable such that the engaged position is configured to engage an inner diameter of a therapy sheath hub.

### CROSS REFERENCE TO RELATED APPLICATIONS

The present application claims priority to and the benefit of U.S. Provisional Patent Application No. 63/616,201, filed Dec. 29, 2023, the entire disclosure of which is incorporated herein by reference.

### TECHNICAL FIELD

The present disclosure relates to medical systems and methods for transseptal puncture in a patient. More specifically, the present disclosure relates to medical systems and methods of delivering the therapeutic device to a target area in a patient.

### BACKGROUND

Treatment of cardiac disease or ailments in the left side of the heart require access form the right atrium (RA) by means of transseptal puncture through the septum to enable the delivery of therapeutic devices. These devices are delivered into the RA through its corresponding delivery sheath. Prior to device delivery, the sheath is used with a corresponding, or kitted, dilator and guidewire to cross the atrial septum defect (ASD) at the transseptal puncture site into the left atrium. Once the sheath is delivered into and positioned within the left atrium, the dilator and guidewire are exchanged for a therapeutic device.

The kitted dilator and guidewire each have a distinct size and are configured to be used with the dilator, guidewire, and therapeutic device of the kit, and generally may not be compatible with dilators, guidewires, and/or therapeutic devices which were not a part of the kit.

### SUMMARY

The present disclosure is directed to an apparatus which may be adjusted to mechanically connect a dilator to delivery sheaths having varying proximal hub sizes and features. Thus, the adapter may be used with various sheaths. The adapter may be formed as a part of a dilator hub, or may be formed as an attachment mechanism to mechanical connection to the dilator hub, such that the adapter and therefore a single the dilator hub, may be used with a multiple of sheaths of varying shapes and sizes.

Example 1 is an adaptable dilator for use with a crossing device, the adaptable dilator comprising: a dilator hub housing defining a distal end and a proximal end, a dilator body extending distally from the dilator hub housing, and a locking feature disposed adjacent the proximal side of the dilator hub housing, wherein the locking feature is movable between a neutral position defining a first diameter and a second position defining a second diameter, wherein the locking featured defines an engaged position between the neutral position and the second position wherein the engaged position of the locking feature defines an engaged diameter, wherein the engaged diameter is variable, such that in the engaged position the locking feature is configured to engage an inner diameter of a therapy sheath hub.

Example 2 is the adaptable dilator of Example 1, wherein the locking feature comprises at least one arm extending from a proximal side of the dilator hub housing wherein the at least one arm extends beyond the proximal side of the dilator hub housing.

Example 3 is the adaptable dilator of Example 2, wherein the at least one arm extends away from the dilator hub housing.

Example 4 is the adaptable dilator of any of Examples 2-3 wherein the at least one arm comprises an arm protrusion on an exterior surface of a distal end of the at least one arm.

Example 5 is the adaptable dilator of Example 1, wherein dilator hub housing further comprises a shaft extension extending from the proximal side, wherein the locking feature is formed integral to the shaft extension.

Example 6 is the adaptable dilator of Example 5, wherein the locking feature is in mechanical connection with a trigger, such that the trigger is configured to move the locking feature between the neutral position and the second position.

Example 7 is the adaptable dilator of any of Examples 5-6, wherein the locking feature is at least two protrusions radially spaced about the shaft extension.

Example 8 is the adaptable dilator of Example 7, wherein the at least two protruding features are diametrically opposed about the shaft extension.

Example 9 is the adaptable dilator of any of Examples 5-8, wherein the locking feature is configured to form a friction fit with the therapy sheath hub.

Example 10 is the adaptable dilator of any of Examples 1-9, wherein the first diameter is larger than the second diameter.

Example 11 is the adaptable dilator of Example 5, wherein the locking feature is an expandable membrane disposed about an exterior surface of the shaft extension.

Example 12 is the adaptable dilator of Example 11, wherein the dilator hub housing further comprises a rotatable dial in mechanical connection with the expandable membrane, such that rotation of the rotatable dial transitions the expandable membrane between the neutral position and the engaged position.

Example 13 is the adaptable dilator of any of Examples 11-12, wherein the first diameter is smaller than the second diameter.

Example 14 is the adaptable dilator of any of Examples 1-13, wherein the locking feature in the engaged position maintains one or more of the axial or radial position of the dilator hub housing.

Example 15 is the adaptable dilator of any of Examples 1-14, wherein the engaged diameter is between 8 French and 18 French.

Example 16 is an adaptable dilator for use with a crossing device, the adaptable dilator comprising: a dilator hub housing defining a distal end and a proximal end, a dilator body extending distally from the dilator hub housing, and a locking feature disposed adjacent the proximal side of the dilator hub housing, wherein the locking feature is movable between a neutral position defining a first diameter and a second position defining a second diameter, wherein the locking featured defines an engaged position between the neutral position and the second position wherein the engaged position of the locking feature defines an engaged diameter, wherein the engaged diameter is variable, such that in the engaged position the locking feature is configured to engage an inner diameter of a therapy sheath hub.

Example 17 is the adaptable dilator of Example 16, wherein the locking feature comprises at least one arm extending from a proximal side of the dilator hub housing wherein the at least one arm extends beyond the proximal side of the dilator hub housing.

Example 18 is the adaptable dilator of Example 17, wherein the at least one arm extends away from the dilator hub housing.

Example 19 is the adaptable dilator of Example 16, wherein dilator hub housing further comprises a shaft extension extending from the proximal side, wherein the locking feature is formed integral to the shaft extension.

Example 20 is the adaptable dilator of Example 19, wherein the locking feature is in mechanical connection with a trigger, such that the trigger is configured to move the locking feature between the neutral position and the second position.

Example 21 is the adaptable dilator of Example 19, wherein the locking feature is at least two protrusions radially spaced about the shaft extension.

Example 22 is the adaptable dilator of Example 16, wherein the at least two extensions are two protruding features diametrically opposed about the shaft extension.

Example 23 is the adaptable dilator of Example 19, wherein the locking feature is configured to form a friction fit with the therapy sheath hub.

Example 24 is the adaptable dilator of Example 16, wherein the first diameter is larger than the second diameter.

Example 25 is the adaptable dilator of Example 19, wherein the locking feature is an expandable membrane disposed about an exterior surface of the shaft extension.

Example 26 is the adaptable dilator of Example 25, wherein the dilator hub housing further comprises a rotatable dial in mechanical connection with the expandable membrane, such that rotation of the rotatable dial transitions the expandable membrane between the neutral position and the engaged position.

Example 27 is the adaptable dilator of Example 25, wherein the first diameter is smaller than the second diameter.

Example 28 is the adaptable dilator of Example 16, wherein the locking feature in the engaged position maintains one or more of the axial or radial position of the dilator hub housing.

Example 29 is the adaptable dilator of Example 16, wherein the engaged diameter is between 8 French and 18 French.

Example 30 is an assembly for facilitating access to a patient's heart, the assembly comprising: an adaptable dilator, the adaptable dilator comprising: a dilator hub housing defining a distal end and a proximal end, a dilator body extending distally from the dilator hub housing, and a locking feature disposed adjacent the proximal side of the dilator hub housing, wherein the locking feature is movable between a neutral position defining a first diameter and a second position defining a second diameter, wherein the locking featured defines an engaged position between the neutral position and the second position, a therapy sheath hub defining an inner diameter, wherein the engaged position of the locking feature defines an engaged diameter, and wherein the engaged diameter is variable, such that in the engaged position the locking feature is configured to engage the inner diameter of the therapy sheath hub.

Example 31 is the assembly of Example 30, wherein the locking feature comprises at least one arm extending from a proximal side of the dilator hub housing wherein the at least one arm extends beyond the proximal side of the dilator hub housing.

Example 32 is the assembly of Example 30, wherein dilator hub housing further comprises a shaft extension extending from the proximal side, wherein the locking feature is formed integral to the shaft extension.

Example 33 is the assembly of Example 32, wherein the locking feature is in mechanical connection with a trigger, such that the trigger is configured to move the locking feature between the neutral position and the second position.

Example 34 is the assembly of Example 32, wherein the locking feature is an expandable membrane disposed about an exterior surface of the protruding feature.

Example 35 is a method of engaging an adaptable dilator and a therapy sheath, the method comprising: providing an adaptable dilator, the adaptable dilator comprising: a dilator hub housing defining a distal end and a proximal end, a dilator body extending distally from the dilator hub housing, and a locking feature disposed adjacent the proximal side of the dilator hub housing, wherein the locking feature is movable between a neutral position defining a first diameter and a second position defining a second diameter, wherein the locking featured defines an engaged position between the neutral position and the second position, providing a therapy sheath hub defining an inner diameter, transitioning the locking feature from the neutral position to the second position, engaging the adaptable dilator with the therapy sheath hub, releasing the locking feature to transition between the second position and the engaged position, wherein in the engaged position the locking feature is engaged with the inner diameter of the therapy sheath hub.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the invention.

FIGS. 2A-2B are perspective views of a dilator and sheath according to embodiments of the invention.

FIG. 3A is a perspective view of a adaptable dilator according to embodiments of the invention.

FIG. 3B is a perspective view of the example adaptable dilator of FIG. 3A in a neutral position according to embodiments of the invention.

FIG. 3C is a perspective view of the example adaptable dilator of FIG. 3A in a second position according to embodiments of the invention.

FIG. 3D is a sectional view of the example adaptable dilator of FIG. 3A engaged with a sheath hub according to embodiments of the invention.

FIG. 4A is a perspective view of another example adaptable dilator in the neutral position according to embodiments of the invention.

FIG. 4B is a perspective view of the example adaptable dilator of FIG. 4A in the second position according to embodiment of the invention.

FIG. 4C is a perspective view of the example adaptable dilator of FIG. 4A engaged with a sheath hub according to embodiments of the invention.

FIG. 5A is a perspective view of another example adaptable dilator in the neutral position according to embodiments of the invention.

FIG. 5B is a sectional of the example adaptable dilator of FIG. 5A in the neutral position according to embodiments of the invention.

FIGS. 5C-D are perspective views of the example adaptable dilator of FIG. 5A in the second position according to embodiment of the invention.

FIG. 6A is a perspective view of another example adaptable dilator in the neutral position according to embodiments of the invention.

FIG. 6B is a sectional view of the example adaptable dilator of FIG. 6A engaged with a sheath hub according to embodiments of the invention.

FIG. 7A is a perspective view of another example adaptable dilator in the neutral position according to embodiments of the invention.

FIG. 7B is a perspective view of the example adaptable dilator of FIG. 7A in the second position according to embodiments of the invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

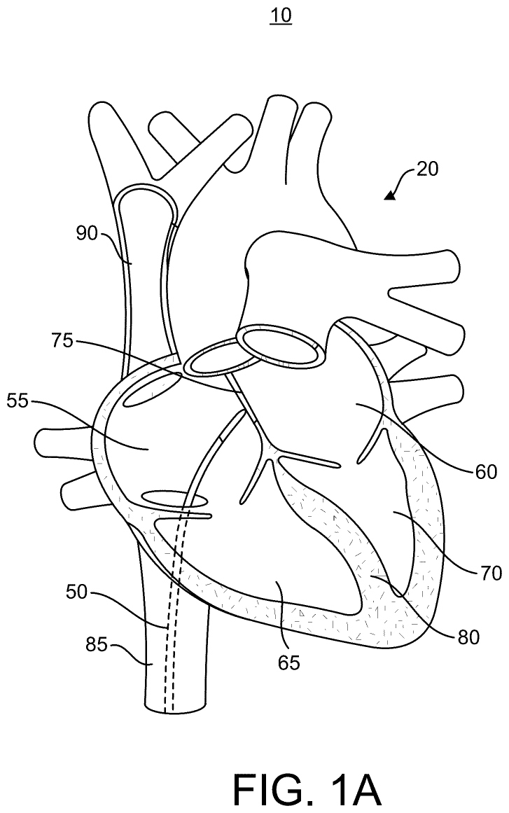

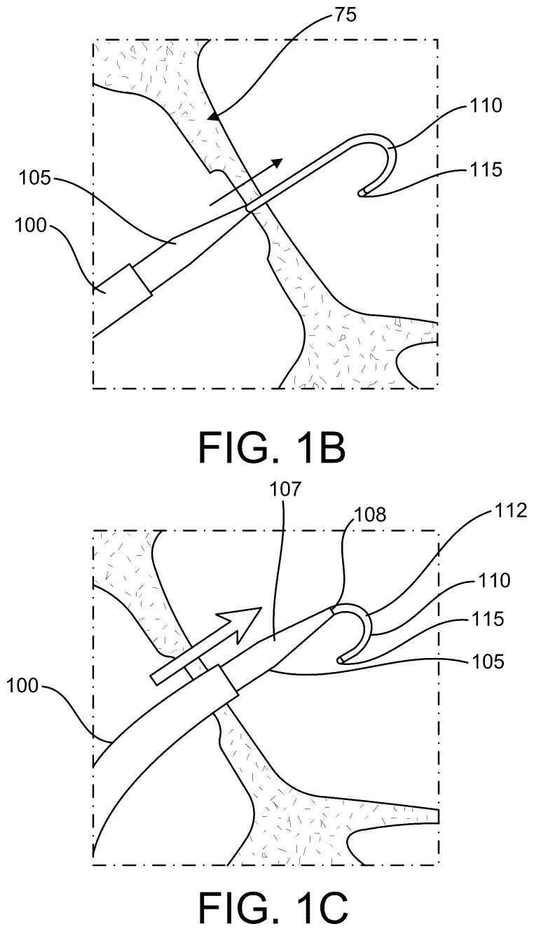

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes a dilator sheath 100, a dilator 105 having a dilator body 107 and a tapered distal tip portion 108, and a perforation device (e.g., a radiofrequency (RF) perforation device) 110 having distal end portion 112 terminating in a tip electrode 115. As shown, in the assembled use state illustrated in FIGS. 1A-1C, the RF perforation device 110 can be disposed within the dilator 105, which itself can be disposed within the dilator sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The dilator sheath 100 may then be introduced into the femoral vein over the guidewire, and advanced towards the heart 20. In one embodiment, the distal ends of the guidewire and dilator sheath 100 are then positioned in the SVC 90. These steps may be performed with the aid of an imaging system, e.g., fluoroscopy or ultrasonic imaging. The dilator 105 may then be introduced into the dilator sheath 100 and over the guidewire, and advanced through the dilator sheath 100 into the SVC 90. Alternatively, the dilator 105 may be fully inserted into the dilator sheath 100 prior to entering the body, and both may be advanced simultaneously towards the heart 20. When the guidewire, dilator sheath 100, and dilator 105 have been positioned in the SVC 90, the guidewire is removed from the body, and the dilator sheath 100 and the dilator 105 are retracted so that their distal ends are positioned in the right atrium 55. The RF perforation device 110 described can then be introduced into the dilator 105, and advanced toward the heart 20. In some embodiments, the guidewire may include the RF perforation device 10.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Boston Scientific Medical Device, LTD. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated kitted dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

In certain embodiments, catheters, therapy devices and sheaths can be deployed through the sheath 100, after it is successfully deployed into the desired heart chamber (e.g., the left atrium). In other embodiments, the therapy device (e.g., mapping catheter, therapy sheath, medical device, etc.) is part of the sheath 100, creating a therapy sheath.

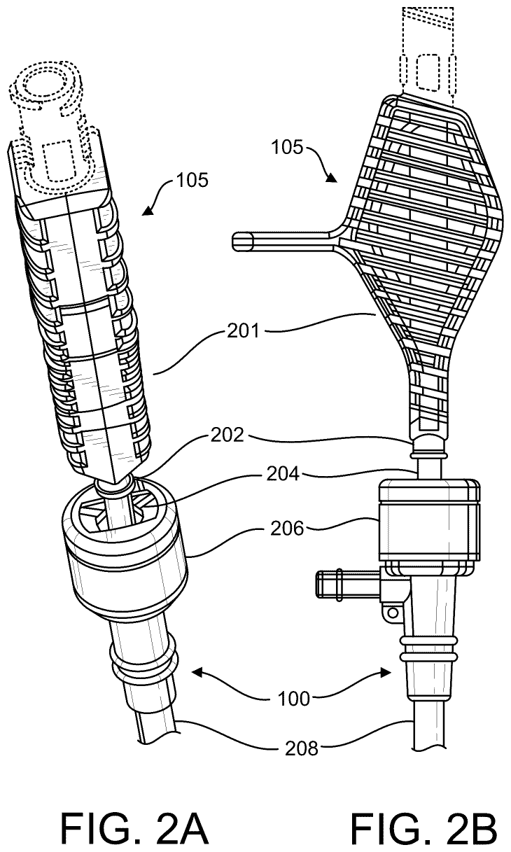

FIGS. 2A-2B are perspective views of a kitted dilator and sheath according to known embodiments of a dilator inserted into a sheath. As shown, the dilator 105 is partially inserted into a lumen of the sheath 100. The dilator 105 includes a handle 201, a dilator hub 202 and a dilator shaft 204, wherein each of the dilator hub 202 and the dilator shaft 204 define fixed diameters. The sheath 100 includes a sheath hub 206 which defines a fixed opening diameter and a sheath body 208. Thus, the dilator hub 202 and dilator shaft 204 are designed for the specific size of the sheath hub 206, as the sheath hub 206 retains and adjoins to the dilator. Additionally, as illustrated, the dilator 105, at least, the handle 201 thereof must be removed prior to inserting or removing additional sheaths, for example, a therapy sheath, which may have a different inner diameter, and may require a separate dilator to hold the sheath in close proximity to the dilator.

The present invention as illustrated in the figures and discussed herein is an adaptable dilator hub which may be connectable to different delivery sheaths with varying proximal hub sizes and features. In this regard, the adaptable dilator hub comprises a locking feature which may transition between a neutral position, and a second position, defining an engaged position therebetween. In the engaged position the adaptable dilator hub may engage with sheath hubs spanning various diameters. The locking feature of the adaptable dilator hub additionally may engage with the sheath hub, to effectively lock the adaptable dilator to the sheath hub. The locking feature may prevent axial and radial rotation with respect to the sheath hub.

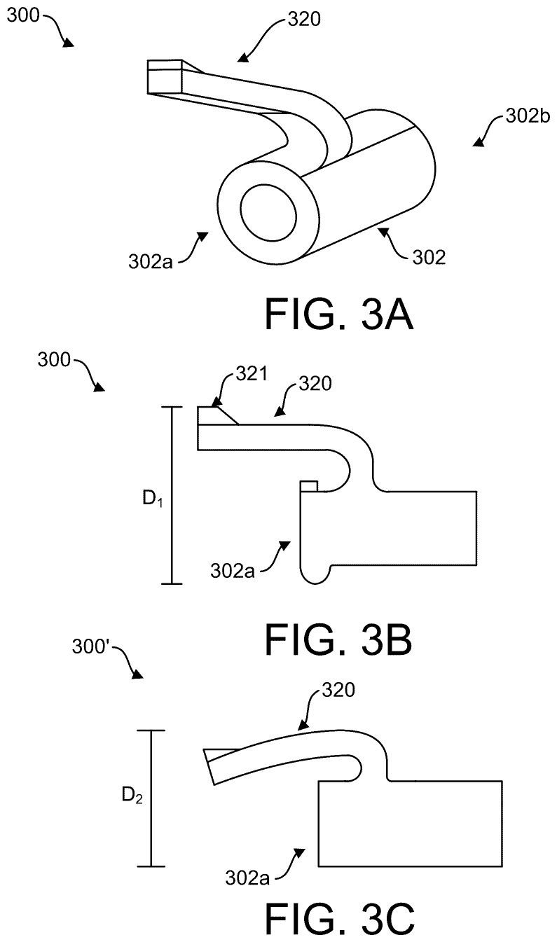

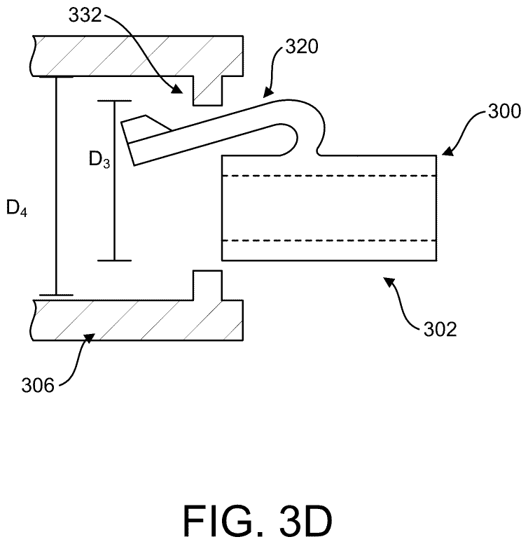

FIGS. 3A-D illustrate a first adaptable dilator 300. The adaptable dilator 300 may comprise a dilator hub housing 302 defining a proximal end 302a and a distal end 302b, wherein the proximal end 302a is opposite the distal end 302b. In some embodiments, the proximal end 302a is configured to engage with a therapy sheath, or therapy hub to couple the adaptable dilator 300 thereto. Further, in some embodiments a dilator body may extend from the distal end 302b of the dilator hub housing 302.

The adaptable dilator 300 may comprise a locking feature to secure the proximal end 302a of the dilator hub housing 302 to a therapy sheath or therapy hub 306. In the present embodiments the locking feature is configured as an arm 320 extending from the proximal end 302a of the dilator hub housing 302. The arm 320 may extend from the dilator hub housing 302 at an angle, such that the arm 320 may be depressed from a neutral position (e.g., FIGS. 3A-B) to a second position (e.g., FIG. 3C).

In some embodiments, the arm 320 may be formed of a flexible material which retains is structure. In this regard, the arm 320 may be bendable, however, the material may retain its neutral state, and be biased to the neutral position.

In some embodiments, the arm 320 may be enhanced to promote engagement with a therapy sheath or therapy sheath hub 306. In some embodiments, the arm 320 may comprise a protrusion 321 extending from an upper surface the arm 320. The protrusion 321 may engage with a feature 332 of the therapy sheath or therapy hub 306 to secure the position of the arm 320 therein. In other embodiments, the arm 320 may comprise an elastomer pad or similar to frictionally engage the internal surface of the therapy sheath or therapy sheath hub 306.

The adaptable dilator 300, illustrated in FIGS. 3A-B, show the arm 320 in a neutral position, wherein there are no forces being applied to the arm 320. Further, in the neutral position the arm 320 may define a first diameter D₁, which extends from the arm 320 to the opposing surface of the dilator hub housing 302. The first diameter D₁, may be larger than a sheath diameter (see e.g., D₄ in FIG. 3D), such that in the neutral position the adaptable dilator 300 may not be inserted into a sheath or sheath hub due to the greater diameter.

In the second position, illustrated in FIG. 3C, the arm 320 may be compressed. In the second position the arm 320 and the dilator hub housing 302 may define a second diameter D₂, wherein the second diameter D₂ is smaller than the first diameter D₁. In this regard, while the adaptable dilator 300 is in the second position, the second diameter D₂ may be sized such that the adaptable dilator 300 may be inserted into a therapy sheath or therapy sheath hub.

In some embodiments, the adaptable dilator 300 may be formed from an elastic material. In this regard the arm 320 may be depressed to the second position to fit into the inner dimeter of the therapy sheath hub or therapy sheath while maintaining the structure and strength of the adaptable dilator 300. Thus, the arm 320 may be designed to be moved between the neutral position and the second position without failing (e.g., cracking), while maintaining a force (e.g., friction or elastic) against the inner surface of the therapy sheath or therapy sheath hub. In some embodiments, a polyethylene material (e.g., HDPE, LDPE, MDPE, etc.), or an acrylonitrile butadiene styrene (ABS) may be used to form the adaptable dilator 300. In some embodiments, other plastics with similar features may be used.

In some embodiments, the arm 320 may be fixed to the dilator hub housing 302, while in other embodiments the arm 320 may be movable along the dilator hub housing 302. In this regard, the dilator hub housing 302 may comprise a slide channel which the arm 320 is positioned and movable within. In another embodiment the dilator hub housing 302 may comprise a sliding moving feature which is moveable along the dilator hub housing 302, wherein the arm 320 is attached to the sliding moving feature. Thus, the position of the arm 320 may be adjusted along the length of the dilator hub housing 302, thereby providing adaptability to therapy sheaths, and therapy sheath hubs of varying sizes and geometries.

FIG. 3D illustrates the adaptable dilator 300 engaged with the therapy sheath hub 306. In some embodiments, the therapy sheath hub 306 may comprise a smooth inner surface, while in other embodiments the therapy sheath hub 306 may comprise one or more surface features 332. In some embodiments, the one or more surface features 332 may comprise a protrusion extending from the inner surface, while in other embodiments the surface feature 322 may be an elastomer pad, or other pad which may increase the friction coefficient at the inner surface.

Upon engagement with the therapy sheath hub 306 the arm 320 of the dilator hub housing 302 may decompress from the second position to an engaged position, as illustrated. In the engaged position the arm 320 and the dilator hub housing 302 may define an engaged diameter D₃. The engaged diameter D₃ may be smaller than the first diameter D₁ and larger than the second diameter D₂. In some embodiments, the engaged diameter D₃ may be about the same size as the inner diameter D₄ of the therapy sheath, or therapy sheath hub. Thus, the engaged diameter D₃ allows the dilator hub housing 302, specifically the arm 320 and the distal end 302a, to be retained within the therapy sheath or the therapy sheath hub 306.

Further, the engagement between the arm 320 and the therapy sheath hub 306 may prevent both axial and radial movement of the adaptable dilator 300 in relation to the therapy sheath hub 306. Additionally, recompressing the arm 320 from the engaged position to the second position may allow for axial and radial movement of the dilator hub housing 302 in relation to the therapy sheath 306. In this regard, when the locking mechanism (e.g., the arm 320) is engaged with the therapy sheath hub 306 the position of the device is maintained along the axial plane and the radial plane, thereby preventing undesirable movement throughout the use of the device 300.

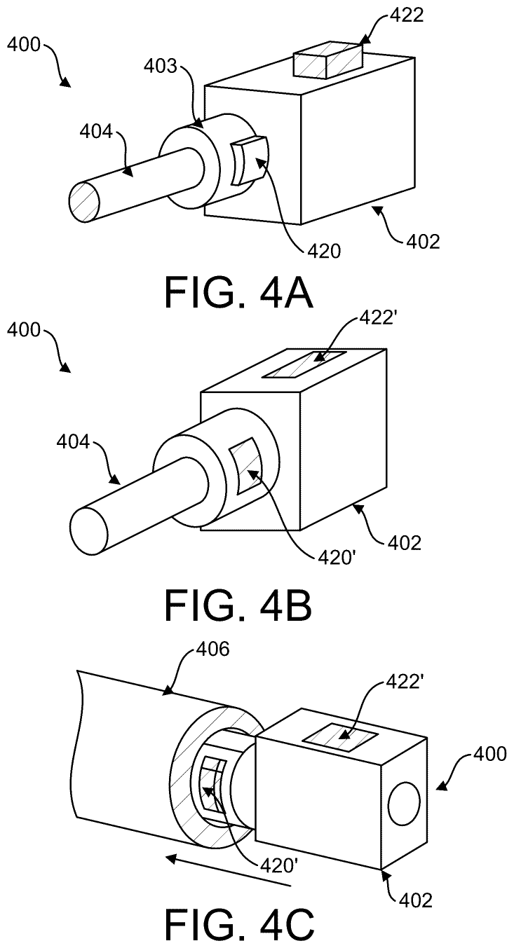

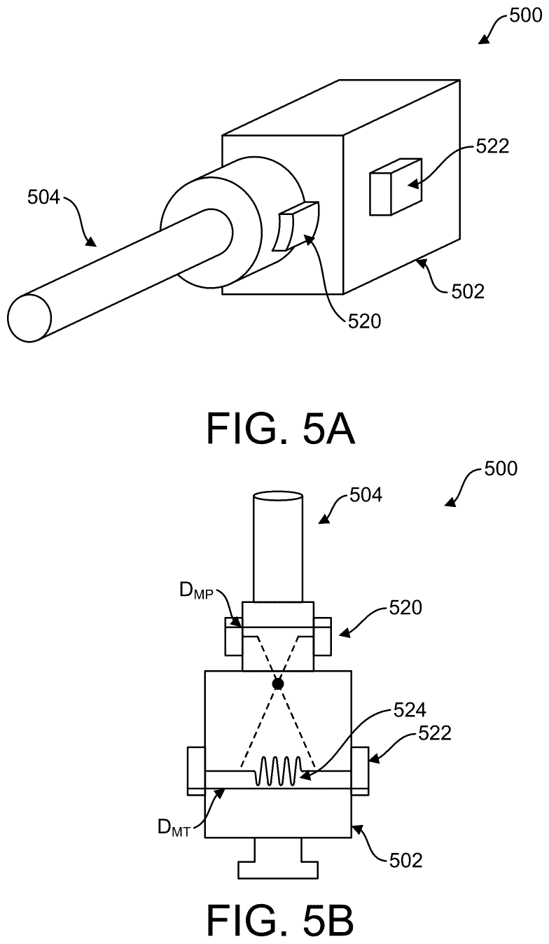

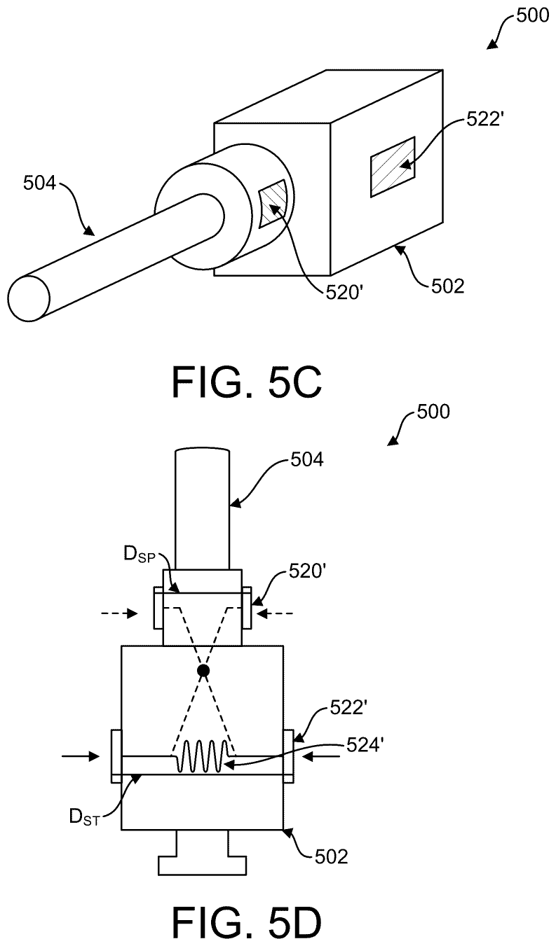

In another embodiment the locking feature may be in mechanical communication with an internal elastic mechanism within the dilator hub housing, as illustrated in FIGS. 4A-5D.

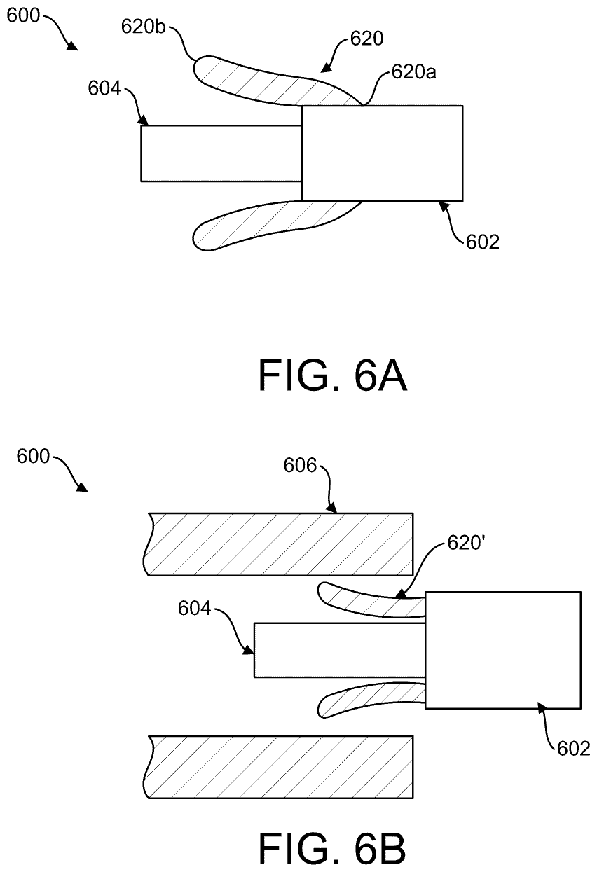

FIGS. 4A-C illustrate an example adaptable dilator 400 defining a dilator hub housing 402 comprising a dilator shaft extension body 403 extending distally from the dilator hub housing 402. Further, a dilator shaft 404 may extend from the dilator shaft extension body 403. In some embodiments, the dilator shaft 404 may extend throughout the dilator hub extension body 403. The dilator hub housing 402 may include a locking mechanism comprising a protruding feature 420 in mechanical communication with a trigger 422. In some embodiments, the trigger 422 may be a single trigger, while in other embodiments, for example as illustrated in FIG. 6A, the trigger may be more than one trigger.

In the example embodiment, the trigger 422 and the protruding feature 420 may be in corresponding positions. In this regard, when the trigger 422 is in a neutral position the protruding feature 420 may also be in a neutral position. Similarly, when the trigger 422′ is in the second position, the protruding feature 422′ may also be in the second position. In some embodiments, the adaptable dilator 400 may be an elastic system. Thus, the action of depressing the trigger 422 may cause the protruding feature 420 to retract in a similar manner.

In the neutral position, each of the trigger 422 and the protruding feature 420 may extend from the dilator hub housing 402 and the dilator shaft extension body 403 respectively. As illustrated in FIG. 5C, the trigger 422′ may be depressed into the second position when the dilator hub housing 402 is inserted into a therapy sheath 406. After insertion the trigger 422′ may be released and the protruding feature 420 may extend from the second position to an engaged position. In the engaged position, as discussed with reference to FIGS. 3A-D, the protruding feature 420′ may be positioned between the second position and the neutral position. Thus, the protruding feature 420 may engage with the therapy sheath 406 thereby creating a connection between the two elements, until the trigger 422 is depressed to the second position thereby releasing the protruding feature 420 from the engaged position to the second position.

In another example embodiment, an example adaptable dilator 500 may comprise more than one protruding feature 520, and more than one trigger 522. In this regard, similar to the example adaptable dilator illustrated in FIGS. 4A-C the triggers 522 may be in mechanical connection with the protruding features 520. In this regard, as discussed previously, when the triggers 522 are in a neutral position, illustrated in FIGs, 5A-B the protruding features 520 are also in the neutral position, and when the triggers 522′ are in the second position, illustrated in FIGS. 5C-D, the protruding features 520′ are also in the second position.

The protruding features 520 may be positioned on a shaft extension body 503 extending from the proximal side of the dilator housing 502. In other embodiments the protruding features 520 may be positioned directly on the dilator shaft 504. In some embodiments, the protruding features 520 may be two protruding features, radially spaced about the shaft extension body 503. In some embodiments, the two protruding features 520 may be diametrically opposed from one another, while in other embodiments, the two protruding features 520 may be unevenly disposed about the shaft extension body 503.

In some embodiments, illustrated in FIGS. 5B, 5D, the mechanical connection between the triggers 522 and the protruding features 520 may be an elastic mechanism 524. In some embodiments, the elastic mechanism 524 may be a spring, such that in the neutral state the protruding features define a first diameter D_{MP}, and the triggers 522 define a first diameter D_{MT}, wherein the first diameter of the protruding features and the triggers are a maximum diameter. When the trigger 522 is depressed to the second position the protruding features 520′ define a second dimeter D_{SP}, and the trigger 522′ defines a second diameter D_{ST}, wherein the second dimeter of the protruding features and triggers is a minimum diameter. The second diameter D_{SP} of the protruding feature 520′ may be smaller than the inner diameter of the therapy sheath and/or therapy sheath hub (e.g., D₄ FIG. 3D). In this regard, the second dimeter of the protruding feature D_{SP} may be less than 8 French, such that the adaptable dilator 500 may engage with any therapy sheath or therapy sheath hub.

After the dilator shaft 504, dilator shaft extension body 503, and the protruding features 520 are inserted into the respective therapy sheath hub and/or therapy sheath, the triggers 522 may be released from the second position to the neutral position. When the elastic mechanism 524 is released, the protruding features 520 may transition to an engaged position wherein the protruding features 520 exhibit an outward force onto the inner surface of the therapy sheath and/or therapy sheath hub. In embodiments where the dilator shaft extension body 503 defines the same shape as the therapy sheath and/or therapy sheath hub a seal may be formed between the protruding features 520 in the engaged position and the inner surface of the therapy sheath/therapy sheath hub. In some embodiments, the protruding feature may be an elastic material such that the elastic material forms a friction fit with the inner surface of the therapy sheath or therapy sheath hub.

In some embodiments, the triggers and the protruding features may be on the same side of the adaptable dilator, while in other embodiments, the triggers and the protruding features may be radially spaced from one another.

In yet another embodiment the adaptable dilator may comprise a locking mechanism configured as multiple side arms extending from the dilator hub housing, as illustrated in FIGS. 6A-B.

In some embodiments, an adaptable dilator 600 may define a dilator hub housing 602 defining a proximal end and a distal end. A dilator shaft 604 may extend from the proximal side of the dilator hub housing 602, and in some embodiments, a dilator body may extend distally from the dilator hub housing 602.

The locking mechanism may be formed as more than one arm 620 extending from the proximal side of the dilator hub housing 602. In this regard, the more than one side arm 620 may extend from a connection point 620a on the dilator hub housing 602 any extend away from the dilator shaft 604. The more than one arm 620 may end in a tip 620b positioned away from the dilator shaft 604 and dilator hub housing 602. In some embodiments, the more than one arm 620 may extend such that in the neutral position the at least one arm 620 is convex between the connection point 620a and the tip 620b. In this regard, the at least one arm 620 may extend partially away from the dilator shaft 604 with a convex bow. In some embodiments, the tip 620b may return towards the dilator shaft 604 in the neutral position, as illustrated in FIG. 6A.

In some embodiments, when the at least one arm 620′ is in second position the at least one arm 620′ may be at least partially concave. In this regard the at least one arm may be flexed towards the dilator shaft 604 between the proximal end of the dilator hub 602 and the tip 620b of the at least one arm 620′.

In some embodiments, the at least one arm 620 may be formed of an elastic material such that that the at least one arm 620 are compressible such as to fit within a therapy sheath or therapy sheath hub 606, and exhibit a frictional force on the therapy sheath hub during engagement. Further, the at least one arm 620 will return to the neutral (e.g., the original shape) after each compression.

In some embodiments, the at least one side arm 620 may be compressed with minimal force, in this regard, the at least one side arm 620′ may be compressed during the insertion into the therapy sheath hub 606, such that no direct contact is necessary between the user and the at least one side arm 620.

In some embodiments, the at least one side arm 620 may be frictionally fit into the sheath hub 606, while in other embodiments, one or more sheath protrusions may be positioned on the interior of the therapy sheath hub 606, so as to retain the at least one side arm 620 within the therapy sheath hub 606.

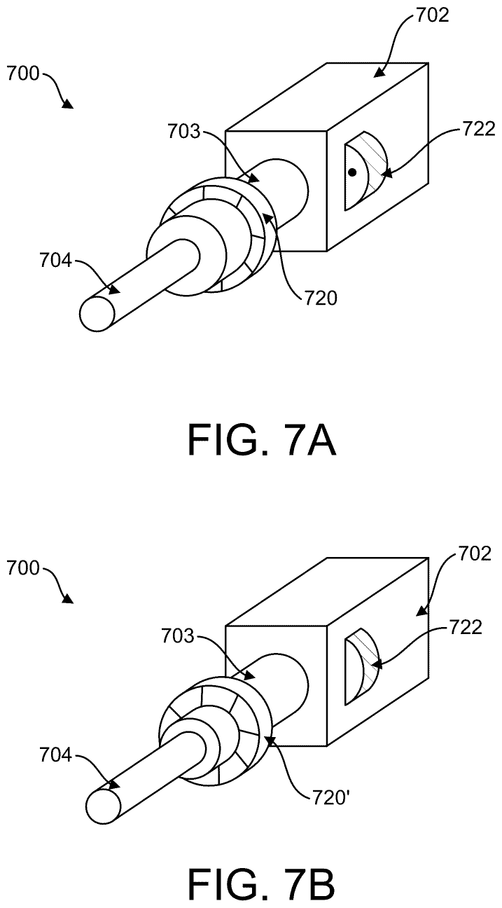

In other embodiments, the locking mechanism may be manually engaged and disengaged. With reference to FIGS. 7A-B an adaptable dilator 700 may comprise a locking feature of an expandable membrane 720 which is manually engaged by a rotational trigger 722. As discussed the adaptable dilator 700 may include dilator hub housing 702, a dilator shaft extension body 703 extending from the proximal side of the dilator hub housing 702 and a dilator shaft 704 extending from the dilator shaft extension body 703. In some embodiments, the expandable membrane 720 may be formed about or integral to the dilator shaft extension 703.

The expandable membrane 720 may be in mechanical connection with the rotational trigger 722, which may be positioned within or integral to the dilator hub housing 702. The rotational trigger 722 may be configured to rotate in a first direction to enlarge the expandable membrane 720 from the neutral position to an engaged position, and may rotate in a second direction, opposite the first direction to retract the expandable membrane 720 to the neutral position.

In the neutral position the expandable membrane 720 may define a first diameter when the expandable membrane 720 is collapsed and retracted and may define a second diameter when the expandable membrane 720 is expanded to a maximum. The expandable membrane 720 may remain in the neutral position during insertion into a therapy sheath. In this regard, the expandable membrane 720 may be expanded to an engaged position when the adaptable dilator is engaged with a therapy sheath.

Each of the adaptable dilators described herein allow for use with different sized therapy sheaths and therapy sheath hubs. To explain, in general the dilator and the therapy sheath hub must be appropriately sized to engage with one another. In contrast the present invention provides flexibility in the size of the therapy sheath to be used by creating a variable diameter of the adaptable dilator.

To explain further, the locking mechanism, when in the engaged position may be adjustable to allow the adaptable dilator to engage with therapy sheaths and therapy sheath hubs of multiple different inner diameters. For example, the first diameter D₁ of the adaptable dilator may define a diameter above 18 French, while the second diameter D₂ may be as small as French. In this regard, the adaptable dilator would be able to engage with a therapy sheath defining an inner diameter between 5 French-18 French.

In addition to the locking mechanism providing an adjustable diameter to engage therapy sheaths, and therapy sheath hubs of varying sizes, the locking mechanism may prevent one or both axial and radial movement of the adaptable dilator in relation to the therapy sheath hub or therapy sheath. In this regard, the adaptable dilator may maintain an orientation and position throughout use.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An adaptable dilator for use with a crossing device, the adaptable dilator comprising:
a dilator hub housing defining a distal end and a proximal end;
a dilator body extending distally from the dilator hub housing; and
a locking feature disposed adjacent the proximal side of the dilator hub housing, wherein the locking feature is movable between a neutral position defining a first diameter and a second position defining a second diameter, wherein the locking featured defines an engaged position between the neutral position and the second position,
wherein the engaged position of the locking feature defines an engaged diameter, wherein the engaged diameter is variable, such that in the engaged position the locking feature is configured to engage an inner diameter of a therapy sheath hub.

2. The adaptable dilator of claim 1, wherein the locking feature comprises at least one arm extending from a proximal side of the dilator hub housing wherein the at least one arm extends beyond the proximal side of the dilator hub housing.

3. The adaptable dilator of claim 2, wherein the at least one arm extends away from the dilator hub housing.

4. The adaptable dilator of claim 1, wherein dilator hub housing further comprises a shaft extension extending from the proximal side, wherein the locking feature is formed integral to the shaft extension.

5. The adaptable dilator of claim 4, wherein the locking feature is in mechanical connection with a trigger, such that the trigger is configured to move the locking feature between the neutral position and the second position.

6. The adaptable dilator of claim 4, wherein the locking feature is at least two protrusions radially spaced about the shaft extension.

7. The adaptable dilator of claim 1, wherein the at least two extensions are two protruding features diametrically opposed about the shaft extension.

8. The adaptable dilator of claim 4, wherein the locking feature is configured to form a friction fit with the therapy sheath hub.

9. The adaptable dilator of claim 1, wherein the first diameter is larger than the second diameter.

10. The adaptable dilator of claim 4, wherein the locking feature is an expandable membrane disposed about an exterior surface of the shaft extension.

11. The adaptable dilator of claim 10, wherein the dilator hub housing further comprises a rotatable dial in mechanical connection with the expandable membrane, such that rotation of the rotatable dial transitions the expandable membrane between the neutral position and the engaged position.

12. The adaptable dilator of claim 10, wherein the first diameter is smaller than the second diameter.

13. The adaptable dilator of claim 1, wherein the locking feature in the engaged position maintains one or more of the axial or radial position of the dilator hub housing.

14. The adaptable dilator of claim 1, wherein the engaged diameter is between 8 French and 18 French.

15. An assembly for facilitating access to a patient's heart, the assembly comprising:
an adaptable dilator, the adaptable dilator comprising:
a dilator hub housing defining a distal end and a proximal end;
a dilator body extending distally from the dilator hub housing; and
a locking feature disposed adjacent the proximal side of the dilator hub housing, wherein the locking feature is movable between a neutral position defining a first diameter and a second position defining a second diameter, wherein the locking featured defines an engaged position between the neutral position and the second position;
a therapy sheath hub defining an inner diameter, wherein the engaged position of the locking feature defines an engaged diameter, and wherein the engaged diameter is variable, such that in the engaged position the locking feature is configured to engage the inner diameter of the therapy sheath hub.

16. The assembly of claim 15, wherein the locking feature comprises at least one arm extending from a proximal side of the dilator hub housing wherein the at least one arm extends beyond the proximal side of the dilator hub housing.

17. The assembly of claim 15, wherein dilator hub housing further comprises a shaft extension extending from the proximal side, wherein the locking feature is formed integral to the shaft extension.

18. The assembly of claim 17, wherein the locking feature is in mechanical connection with a trigger, such that the trigger is configured to move the locking feature between the neutral position and the second position.

19. The assembly of claim 17, wherein the locking feature is an expandable membrane disposed about an exterior surface of the protruding feature.

20. A method of engaging an adaptable dilator and a therapy sheath, the method comprising:
providing an adaptable dilator, the adaptable dilator comprising:
a dilator hub housing defining a distal end and a proximal end;
a dilator body extending distally from the dilator hub housing; and
a locking feature disposed adjacent the proximal side of the dilator hub housing, wherein the locking feature is movable between a neutral position defining a first diameter and a second position defining a second diameter, wherein the locking featured defines an engaged position between the neutral position and the second position;
providing a therapy sheath hub defining an inner diameter;
transitioning the locking feature from the neutral position to the second position;
engaging the adaptable dilator with the therapy sheath hub;
releasing the locking feature to transition between the second position and the engaged position, wherein in the engaged position the locking feature is engaged with the inner diameter of the therapy sheath hub.
